(19) United States
(12) Patent Application Publication
(10) Pub. No.: US 2004/0236277 A1
(43) Pub. Date: Nov. 25, 2004

(54) Device for performing anastomoses

(76) Inventor: Henri Julien Ronsse, Saint-Hilaire St-Mesmin (FR)

Correspondence Address: WENDEROTH, LIND & PONACK, L.L.P., 2033 K STREET N. W., SUITE 800, WASHINGTON, DC, 20006-1021, US

(21) Appl. No.: 10/790,133

(22) Filed: Mar. 2, 2004

(30) Foreign Application Priority Data
Mar. 3, 2003 (FR) 0302567

(51) Int. Cl. A61M 29/00

(52) U.S. Cl. 604/101.01; 606/191; 606/194; 604/96.01

(57) ABSTRACT

The device for performing anastomoses comprises a sheath; a proximal inflatable balloon; and a distal inflatable balloon secured to the sheath, with an independent inflation duct for each balloon. Each inflation duct is connected to the associated balloon and presents a connection coupling. The inflation ducts are secured to the sheath along respective sheath-linked segments extending from the associated balloon towards the proximal end of the sheath, at least beyond the proximal balloon. The sheath-linked segment of the inflation duct for the proximal balloon extends towards the proximal end of the sheath beyond the sheath-linked segment of the inflation duct of the distal balloon.

The present invention relates to a device for performing anastomoses, the device being of the type comprising:

a sheath having a proximal end and a distal end;

at least a proximal inflatable balloon and a distal inflatable balloon secured to the sheath and spaced apart from each other along the length of the sheath; and

an independent inflation duct for each balloon, each inflation duct having a first end connected to the associated balloon and having another end presenting a connection coupling, the inflation ducts being secured to the sheath along respective sheath-linked segments extending from the associated balloon towards the proximal end of the sheath, at least beyond the proximal balloon.

### BACKGROUND OF THE INVENTION

Such a device is described in patent application FR-A-2 803 532.

When the device is in use, the sheath segment containing the balloons is received inside a duct for treatment in an organism. Thus, the balloons and the associated segments of inflation ducts connected to the balloons are not visible to the practitioner.

Only the ends of the inflation ducts fitted with the coupling endpieces are visible, since they project outside the duct for treatment of an organism.

In order to enable the practitioner to distinguish between the two inflation ducts, and associate each of them with one particular balloon, the visible portions of the ducts are identified by color marking.

Nevertheless, in order to inflate or deflate a given balloon, the practitioner must remember which balloons are controlled by inflation ducts marked in which colors.

Such devices are often used during stages of a surgical operation that are critical, which means that the practitioner must perform the operation as quickly as possible. Haste can lead to the surgeon confusing inflation ducts. The wrong balloon is then accidentally inflated or deflated, which can put the patient's health in peril.

### OBJECTS AND SUMMARY OF THE INVENTION

An object of the invention is to propose a device for performing anastomoses which avoids the risk of confusion between the two balloon inflation ducts.

To this end, the invention provides a device for performing anastomoses of the above-specified type, wherein the sheath-linked segment of the duct for inflating the proximal balloon extends along the sheath towards the proximal end of the sheath beyond the sheath-linked segment of the duct for inflating the distal balloon.

In particular embodiments, the device further comprises one or more of the following characteristics:

each inflation duct has a free segment beyond its sheath-linked segment, extending away from the sheath;

the proximal end of the sheath carries a hemostatic valve;

each inflation duct is fitted with a stop cock at its end presenting a connection coupling;

the device includes an intermediate inflatable balloon secured to the sheath between the proximal inflatable balloon and the distal inflatable balloon, together with an inflation duct for inflating the intermediate balloon, which inflation duct has a first end connected to the intermediate balloon and has an opposite end presenting a connection coupling, said duct being secured to the sheath along a sheath-linked segment extending from the intermediate balloon towards the distal end of the sheath to at least beyond the proximal balloon; and the link segment of the duct for inflating the intermediate balloon extends along the length of the sheath beyond the link segment of the duct for inflating the distal balloon and it terminates before the distal end of the link segment of the duct for inflating the proximal balloon;

the diameter of the sheath lies in the range 1.8 millimeters (mm) to 3 mm;

the distance between the distal and proximal balloons lies in the range 40 mm to 80 mm; and

the sheath includes at least one lateral orifice located between the proximal balloon and the proximal end of the sheath.

### BRIEF DESCRIPTION OF THE DRAWINGS

The invention will be better understood on reading the following description given purely by way of example and made with reference to the drawings, in which:

FIG. 1 is an elevation view of a surgical kit including a device of the invention for performing anastomosis;

FIG. 2 is a perspective view showing the use of the device for performing anastomosis of the invention; and

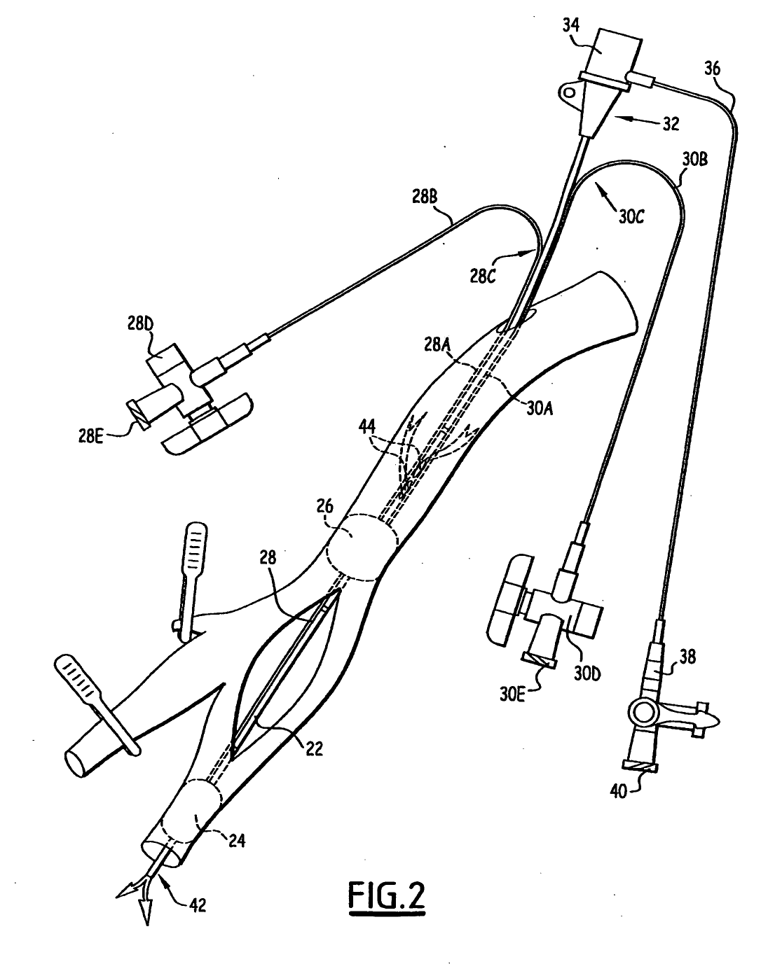

FIG. 3 is a perspective view of a variant embodiment of a device of the invention for performing anastomosis.

### MORE DETAILED DESCRIPTION

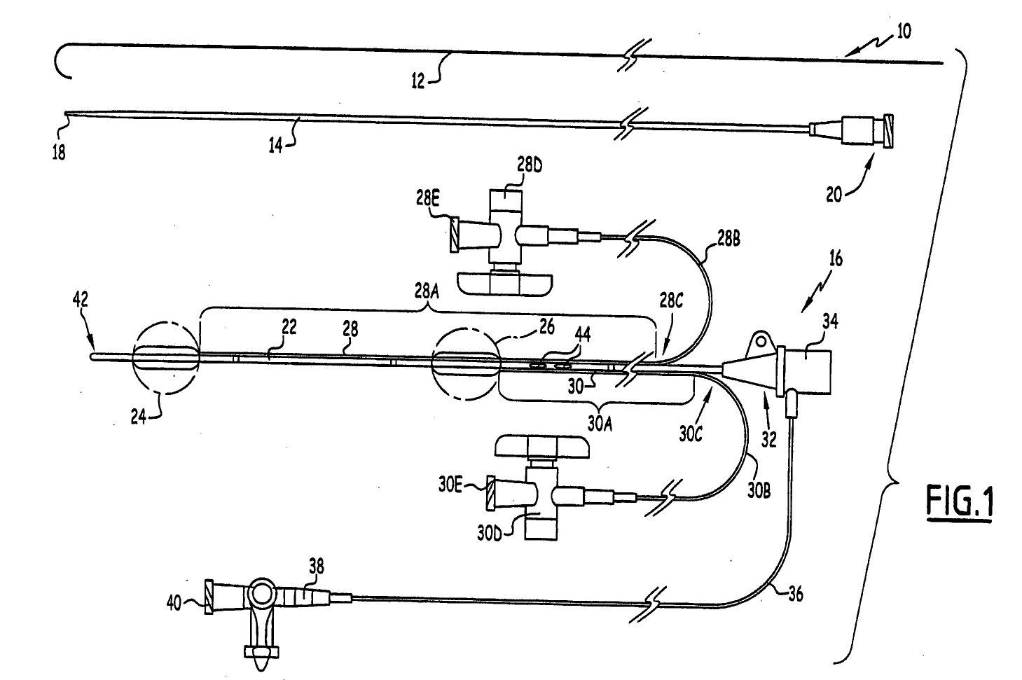

The kit 10 shown in FIG. 1 is for use by a surgeon in performing anastomosis on a duct in an organism, e.g. an artery.

The various elements of the kit are, for example, sold in a common sealed pouch defining a sterile space.

The kit comprises a surgical guide 12, a dilator 14, and a device 16 for performing anastomosis.

The surgical guide 12 is, for example, a J-guide presenting an end curved like a fish hook. The guide is made of flexible steel having a diameter of 0.35 mm, for example, and a length of about 50 centimeters (cm).

The dilator 14 is constituted by a tube of plastics material such as Teflon with an outside diameter of about 2.2 mm and a length of about 24 cm. The inside diameter of the dilator is sufficient to allow the guide 12 to slide therein.

At its distal end, the dilator presents a chamfered tip 18. At its proximal end it has an endpiece 20 for handling and connection purposes.

The device 16 for performing anastomosis essentially comprises a sheath 22 having two inflatable balloons 24 and 26 secured thereto, each connecting to a respective inflation duct 28, 30.

More precisely, the sheath 22 is formed of a tube of plastics material such as Teflon. The tube has a total length of about 20 cm. Its outside diameter lies in the range 1.8 mm to 3 mm. Its inside diameter is sufficient to pass the dilator 14.

The proximal end 32 of the sheath 22 presents a hemostatic valve 34. The hemostatic valve 34 enables thread-like elements to be inserted into the sheath 22 from its distal end without blood flowing out. Such thread-like elements are constituted, for example, by the guide 12 and/or the expander 14.

In addition, a flexible tube 36 is connected to the valve 34 to enable fluid to be introduced into the sheath 22. At its free end, the tube 36 is equipped with a stop cock 38, itself fitted with a coupling endpiece 40 for coupling to a source of liquid such as a syringe.

The end of the sheath 22 remote from the hemostatic valve 34 constitutes a distal end referenced 42 and suitable for being inserted in the organic duct to be treated.

The sheath 28 is open at its proximal end to allow blood to flow.

The distal balloon 24 is secured in the vicinity of the distal end 42. It is connected to one end of the inflation duct 28. This duct is secured along the sheath 22 and extends along said sheath from the distal balloon 24 towards the proximal end 32 of the sheath.

The proximal balloon 26 is also secured to the sheath 22 at a distance from the distal balloon 24. These balloons are spaced apart by a distance lying in the range 40 mm to 80 mm, and preferably in the range 45 mm to 60 mm. The inflation duct 30 is connected at a first end to the balloon 26 and it extends along the sheath 22 towards the proximal end 32 thereof.

Each balloon is deformable between a deflated state shown in continuous lines in FIG. 1 and an inflated state shown in chain-dotted lines in said figure.

Between the proximal balloon 26 and the proximal end 32 of the sheath there are formed one or more (e.g. two) oblong orifices 44 through the side wall of the sheath.

The ducts 28 and 30 are linked to the sheath along its length beyond the orifices 44.

In the invention, the sheath-linked segment referenced 30A of the inflation duct 30 extends towards the proximal end 32 of the sheath, beyond a sheath-linked segment referenced 28A of the duct 28. Each of the segments 28A and 30A of the ducts is extended by a respective flexible segment 28B, 30B going away from the sheath 22. The separation points referenced 28C and 30C between the sheath-linked segment of each duct and its free segment are disposed along the sheath 22 in the same order as the associated balloons 24, 26 are themselves disposed along the sheath.

In other words, the separation point 30C of the inflation duct for the proximal end 26 is closer to the proximal end 32 of the sheath than is the separation point 28C of the inflation duct for the distal balloon 24.

At its free end, each of the inflation ducts presents a respective stop cock 28D, 30D fitted with an endpiece 38E, 30E for connection to a source of fluid for inflating a balloon.

The device of the invention is used as follows.

In conventional manner, the device for performing anastomoses is inserted into the duct to be treated through an opening made in the wall of the duct. The balloons are inflated on either side of the region that is to be incised. Blood flow is then diverted via the sheath 22, penetrating into it through the lateral orifices 44 and leaving from the distal end 42 of the sheath.

It will be understood that during the operation, the separation points 28C, 30C between the sheath and the inflation ducts are visible, since they are situated outside the duct to be treated. Insofar as the order of the separation points along the length of the sheath is the same as the order of the associated balloons, the practitioner can easily determine which duct feeds the distal balloon and which duct feeds the proximal balloon, since the ducts separate from the sheath in the same order as that in which the balloons connected to the ducts are themselves disposed.

Thus, the risks of confusing the inflation ducts are avoided, and the practitioner no longer needs to associate the ducts with the balloons they control, e.g. by means of a color code.

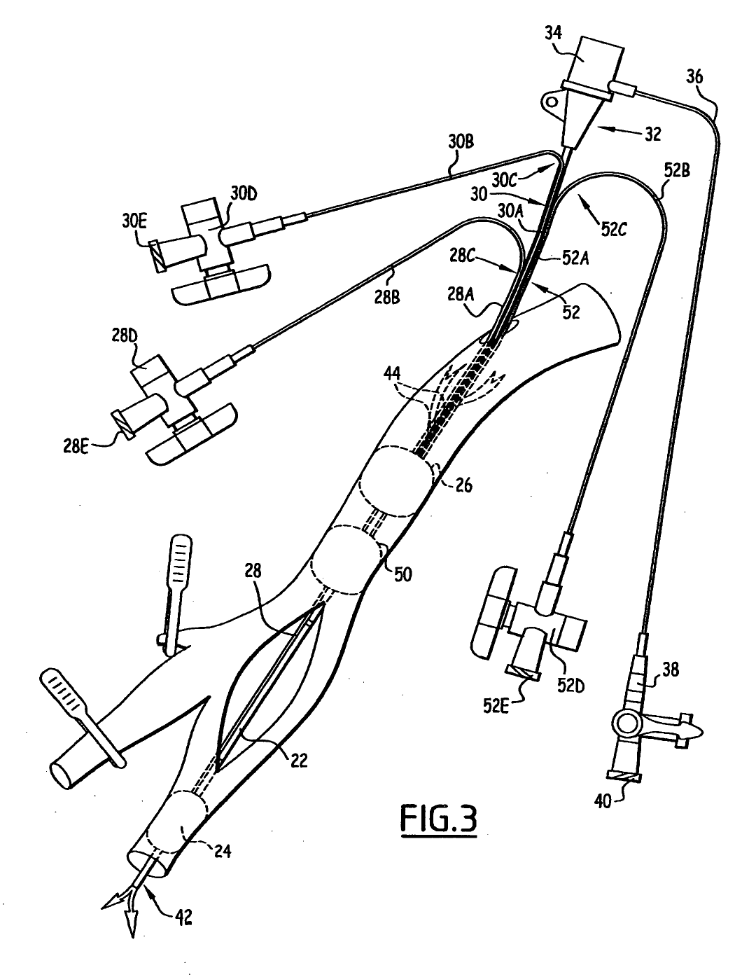

In another embodiment shown in FIG. 3, the device has a third balloon 50 secured to the sheath 22 between the distal balloon 24 and the proximal balloon 26. This intermediate balloon is close to the proximal balloon 26.

An inflation duct 52 is connected at one end to the intermediate balloon 50. At its second end it presents a stop cock 50D fitted with a connection endpiece 50E.

As before, the duct for inflating the intermediate balloon presents a segment 52A that is secured along the heath 22. This segment extends from the intermediate balloon 50 towards the proximal end of the sheath. It is extended in turn by a free segment 52B that goes away from the sheath 22.

The sheath-linked segment 52A of the duct for inflating the intermediate balloon extends beyond the sheath-linked segment 28A of the duct for inflating the distal balloon 24, but stops before the sheath-linked segment 30A of the duct for inflating the proximal balloon 26. Thus, the point of separation referenced 52C of the duct for inflating the intermediate balloon 50 is situated between the separation points 28C and 30C of the inflation ducts for the distal and proximal balloons.

In this embodiment likewise, the practitioner can easily determine which duct feeds which balloon, the ducts being connected to the sheath along the length of the sheath in the same order as the associated balloons are disposed along the length of the sheath.

The intermediate balloon in the example described is disposed beside the proximal balloon 26.

In a variant, it could be disposed close to the distal balloon 24 or at any other location along the length of the sheath as a function of the application in consideration.

The third balloon provides additional safety when clamping large arteries (e.g. the aorta), where the risk of a balloon rupturing could imperil the health of the patient.

What is claimed is:

1. A device for performing anastomoses, the device comprising:
a sheath having a proximal end and a distal end;
at least a proximal inflatable balloon and a distal inflatable balloon secured to the sheath and spaced apart from each other along the length of the sheath; and
an independent inflation duct for each balloon, each inflation duct having a first end connected to the associated balloon and having another end presenting a connection coupling, the inflation ducts being secured to the sheath along respective sheath-linked segments extending from the associated balloon towards the proximal end of the sheath, at least beyond the proximal balloon,
wherein the sheath-linked segment of the duct for inflating the proximal balloon extends along the sheath towards the proximal end of the sheath beyond the sheath-linked segment of the duct for inflating the distal balloon.

2. A device according to claim 1, wherein each inflation duct has a free segment beyond its sheath-linked segment, extending away from the sheath.

3. A device according to claim 1, wherein the proximal end of the sheath carries a hemostatic valve.

4. A device according to claim 1, wherein each inflation duct is fitted with a stop cock at its end presenting a connection coupling.

5. A device according to claim 1, including an intermediate inflatable balloon secured to the sheath between the proximal inflatable balloon and the distal inflatable balloon, together with an inflation duct for inflating the intermediate balloon, which inflation duct has a first end connected to the intermediate balloon and has an opposite end presenting a connection coupling, said duct being secured to the sheath along a sheath-linked segment extending from the intermediate balloon towards the distal end of the sheath to at least beyond the proximal balloon; and
wherein the link segment of the duct for inflating the intermediate balloon extends along the length of the sheath beyond the link segment of the duct for inflating the distal balloon and it terminates before the distal end of the link segment of the duct for inflating the proximal balloon.

6. A device according to claim 1, wherein the diameter of the sheath lies in the range 1.8 mm to 3 mm.

7. A device according to claim 1, wherein the distance between the distal and proximal balloons lies in the range 40 mm to 80 mm.

8. A device according to claim 1, wherein the sheath includes at least one lateral orifice located between the proximal balloon and the proximal end of the sheath.
